(19) United States
(12) Patent Application Publication
(10) Pub. No.: US 2004/0110776 A1
(43) Pub. Date: Jun. 10, 2004

(54) Use of propentofylline to control intraocular pressure

(76) Inventors: Iok-hou Pang, Grand Prairie, TX (US); Mark R Hellberg, Highland Village, TX (US); Abbott F Clark, Arlington, TX (US); Peggy E Hellberg, Highland Village, TX (US); Debra L Fleenor, Benbrook, TX (US)

Correspondence Address: ALCON RESEARCH, LTD., R&D COUNSEL, Q-148, 6201 SOUTH FREEWAY, FORT WORTH, TX, 76134-2099, US

(21) Appl. No.: 10/250,326

(22) PCT Filed: Feb. 22, 2002
(86) PCT No.: PCT/US02/05456

(51) Int. Cl. A61K 31/522

(52) U.S. Cl. 514/263.35

(57) ABSTRACT

Methods for controlling intraocular pressure with propentofylline are disclosed.

The present invention is directed to the use of propentofylline for controlling intraocular pressure (IOP), particularly the elevated intraocular pressure associated with glaucoma and/or ocular hypertension.

### BACKGROUND OF THE INVENTION

Glaucoma is an ocular disease associated with optic nerve head degeneration and loss of vision, which may lead to irreversible blindness. Elevated IOP (ocular hypertension) is a major risk factor of glaucoma. IOP is regulated by the balance between the rate of aqueous humor production from the ciliary epithelium and the rate of its outflow through the trabecular meshwork (TM) and the uveal scleral pathway. In primary open angle glaucoma, the abnormal increase in IOP is mainly due to pathological changes in the TM leading to a significant reduction of outflow facility (Langham, The physiology and pathology of the intraocular pressure. In: Bellows, ed. Glaucoma: Contemporary international concepts. New York: Masson Publishing, 1979:24-48; Segawa, Electron microscopic changes in the trabecular tissue in primary open angle glaucoma. In: Bellows, ed. Glaucoma: Contemporary international concepts. New York: Masson Publishing, 1979:17-23; Rohen, Why is intraocular pressure elevated in chronic simple glaucoma? Anatomical consideration, Ophthalmology 1983;90:758-765).

It has been reported that the resistance of aqueous humor outflow through the TM is normally regulated in part by the ongoing extracellular matrix turnover in the TM (Bradley, et al., Effective matrix metalloproteinases activity on outflow in perfused human organ culture, Investigative Ophthalmology & Visual Science, 1998;39:2649-2658). Matrix metalloproteinases (MMPs) have been proposed as important enzymes regulating the turnover of extracellular matrix in the TM (Alexander, et al., Expression of matrix metalloproteinases and inhibitor by human trabecular meshwork, Investigative Ophthalmology & Visual Science, 1991;32:172-180.1991; Acott, Trabecular extracellular matrix regulation. In: Drance, Van Buskirk, & Neufeld, eds. Pharmacology of Glaucoma. Baltimore: Williams & Wilkins, 1992:125-127.1992; Samples, et al., Regulation of the levels of human trabecular matrix metalloproteinases and inhibitor by interleukin-1 and dexamethasone, Investigative Ophthalmology & Visual Science, 1993;34:3386-3395). Activation of these enzymes could theoretically reduce the excessive and congestive extracellular matrix in the glaucomatous eye and in turn decrease fluid resistance of the outflow pathway. Indeed, when purified metalloproteinases (MMP-2, MMP-3 and MMP-9) were used to perfuse the human anterior segment, outflow facility increased by more than 50% lasting for at least 5 days (Bradley, et al., Effective matrix metalloproteinases activity on outflow in perfused human organ culture, Investigative Ophthalmology & Visual Science, 1998;39:2649-2658). Similarly, perfusion of the anterior segment with interleukin-1α, a cytokine known to increase the expression of matrix metalloproteinases in the TM, also augmented the outflow facility (Bradley, et al., Effective matrix metalloproteinases activity on outflow in perfused human organ culture, Investigative Ophthalmology & Visual Science, 1998;39:2649-2658). In contrast, metalloproteinase inhibitors, whether peptides (such as tissue inhibitor of metalloproteinase) or non-peptides (such as minocylcine, L-tryptophan hydroxamate), suppressed aqueous outflow. Furthermore, Acott, et al. (U.S. Pat. No. 5,260,059) have disclosed a method for treating open-angle glaucoma with a substance to modulate the ratio of matrix metalloproteinases (MMP) to tissue inhibitor of metalloproteinase (TIMP) or MMP/TIMP. The inventors' most preferred substances to achieve this include matrix metalloproteinases, see Column 3, lines 33-37.

Propentofylline (HWA 285; 3-methyl-1-(5′-oxohexyl)-7-propyl-xanthine) is a peripheral vasodilator. It is disclosed in U.S. Pat. No. 4,289,776 (Mohler, et al.) as useful for treating arterial blood flow disturbance or vascular dilatory insufficiency; in U.S. Pat. No. 4,636,507 (Sheetz) for treating host defense mechanisms against trauma; in U.S. Pat. No. 5,310,666 (Aretz, et al.) for the treatment of peripheral, cerebral, and ocular vascular disorders; and in U.S. Pat. No. 4,719,212 (Goto, et al.) for treating cerebral disturbance; and in U.S. Pat. No. 5,762,953 (Venkateshwaran) for treating Alzheimers disease; and in U.S. Pat. No. 5,409,935 (Schubert, et al.) for treating secondary nerve cell damage and functional disorders after cranio-cerebral traumas; and in U.S. Pat. No. 6,037,347 (Schubert, et al.) for treating dementia.

In U.S. Pat. No. 5,780,450 (Shade), adenosine uptake inhibitors are disclosed as being useful for treating retinal and optic nerve head damage following acute or chronic glaucoma, edema, ischemia, hypoxia, or trauma. The adenosine uptake inhibitors disclosed in the patent (including propentofylline) inhibit the uptake or re-absorption of adenosine into the neural cells of ocular tissues and thus help protect the neural cells from damage triggered by or resulting from the above-referenced conditions. The use of adenosine uptake inhibitors for lowering or controlling IOP is not disclosed or suggested. Propentofylline has also been determined to be a neurotrophic factor stimulator which could be useful to treat ophthalmic neurodegeneration resulting from various conditions, including glaucoma (WO/00/32197, Alcon Laboratories, Inc.)

None of the above publications disclose the use of propentofylline for lowering or controlling IOP, or its effect on the regulation of MMP expression in ocular tissues.

Pentoxifylline is a close analog of propentoflline. Topical instillation of 2% pentoxifylline was reported to produce a slight decrease in IOP in ocular normotensive rabbits (Hariton, Ocular hypotension induced by topical dopaminergic drugs and phosphodiesterease inhibitors, European Journal of Pharmacology, 1994;258:85-94). This manuscript also describes the IOP-lowering effect of pentoxifylline when it was administered in combination with other compounds such as 3-(3-hydroxyphenyl)-N-n-propylpiperidine, and trifluperidol. However, propentoflline was not studied or is mentioned in this report.

### SUMMARY OF THE INVENTION

The present invention is directed to methods for controlling intraocular pressure in humans with propentofylline.

### DESCRIPTION OF THE PREFERRED EMBODIMENTS

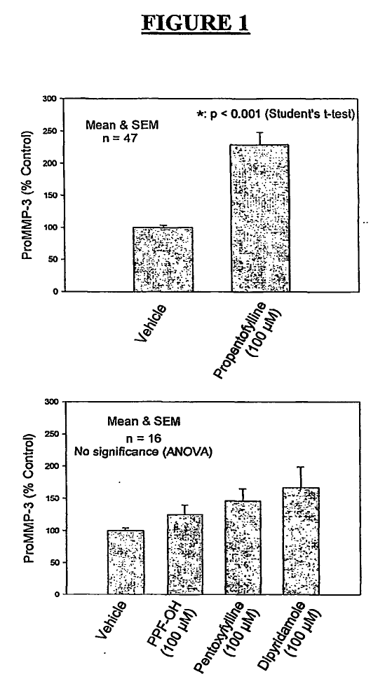

Propentofylline was unexpectedly discovered to stimulate the expression of metalloproteinase-3 (MMP-3; stromelysin) in cultured human trabecular meshwork cells. Incubation of the cells with 100 μM propentofylline for 72 hours significantly increased the expression of MMP-3 (FIG. 1, upper panel). This unexpected effect was unique to propentofylline, since other adenosine reuptake inhibitors and close chemical analogs of propentofylline, such as dipyridamole, pentoxifylline, and hydroxy-propentofylline, did not produce a statistically significant change in MMP-3 expression in the TM cells (FIG. 1, lower panel). This stimulatory effect unique to propentofylline on MMP-3 production by the TM cells suggests that it may modulate the aqueous hydrodynamics in the eye and affect IOP.

Despite the lack of prior evidence that propentofylline lowers IOP, we found that this compound unexpectedly increases the aqueous outflow facility in the human ocular perfusion organ culture. In this ex vivo study, the anterior segments of non-glaucomatous donor eyes were perfused continuously with 100 μM of propentofylline. On days 2 and 3 after the initiation of perfusion, the outflow rates of eyes receiving propentofylline were significantly increased when compared to vehicle-treated eyes (Example 2).

When propentofylline was tested in the rabbit for its potential effect on IOP. It did not produce a statistically significant change (Table 1). In this study, the rabbits were placed in restrainers, and IOP was determined with an Alcon Pneumatonometer after light corneal anesthesia with 0.1% proparacaine (Alcaine® diluted with physiogical saline). Following each IOP measurement, residual anesthetic was washed away with saline. After two baseline measurements, animals were dosed topically on the cornea with vehicle or propentofylline (2×25 μL, 1% solution). IOP measurements were taken at indicated intervals. This finding indicates that topical administration of 500 μg propentofylline onto the eye did not affect the IOP of rabbits. The compound was well tolerated in this study.

TABLE 1
Lack of effect of Propentofylline (500 μg) in Rabbit IOP

| | | |
|---|---|---|
| Time after | IOP (mmHg) | % Change in IOP |

| Treatment (hour) | Mean | SEM | Mean | SEM |
|---|---|---|---|---|
| 0 | 23.5 | 0.9 | 0.0 | 0.0 |
| 1 | 21.0 | 0.7 | −10.0 | 3.0 |
| 3 | 23.6 | 0.9 | 1.3 | 4.7 |
| 5 | 23.5 | 0.3 | 1.1 | 3.4 |
| 7 | 24.0 | 0.5 | 4.7 | 6.2 |

Note:
n = 10. No statistical significance observed.

In the same study, it was also found that similar to propentofylline, pentoxifylline did not significantly lower IOP in the rabbit (Table 2).

TABLE 2
Lack of effect of Pentoxifylline (500 μg) in Rabbit IOP

| | | |
|---|---|---|
| Time after | IOP (mmHg) | % Change in IOP |

| Treatment (hour) | Mean | SEM | Mean | SEM |
|---|---|---|---|---|
| 0 | 23.8 | 0.8 | 0.0 | 0.0 |
| 1 | 21.6 | 1.0 | −8.9 | 2.8 |
| 3 | 23.1 | 0.4 | −1.8 | 3.8 |
| 5 | 27.8 | 0.6 | 17.5 | 3.6 |
| 7 | 27.3 | 0.6 | 15.9 | 5.2 |

Note:
n = 10. No statistical significance observed.

IOP responses in the rabbit eye to various pharmacological agents do not always correspond to IOP responses in primates. For example, clinically active IOP-lowering compounds, such as latanoprost or pilocarpine, had no measurable effect on IOP in the rabbit (Dinslage, et al., Intraocular pressure in rabbits by telemetry II: effects of animal handling and drugs, Investigative Ophthalmology & Visual Science, 1998;39:2485-2489; Van Bijsterveld, et al., The effect of hypotensive drugs on the intraocular pressure after waterloading in rabbits, Documenta Ophthalmologica, 1981;52:189-198). Hence, experimental data regarding IOP obtained from the rabbit cannot be generalized to predict pharmacological actions in primates, including humans.

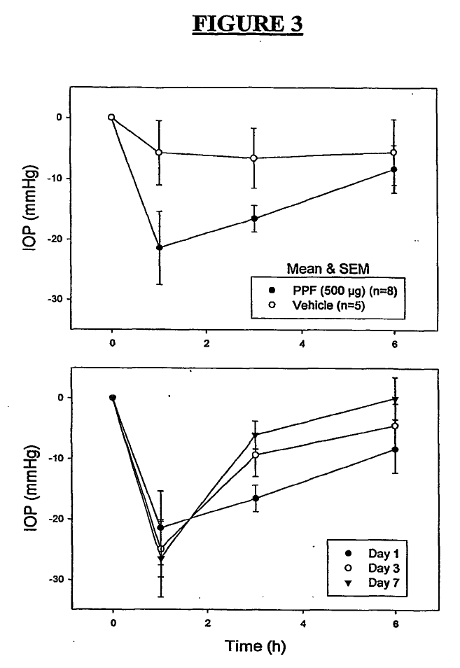

The IOP-lowering effect of propentofylline in primates was discovered when tested in ocular hypertensive monkeys. In this animal model, the IOP of cynomolgus monkeys was artificially elevated by laser-induced photocoagulation of the TM. When 500 μg of propentofylline in aqueous solution was instilled onto the surface of the lasered eye of unanesthetized monkeys, it caused a dramatic decrease in IOP (FIG. 3, upper panel). This IOP-lowering effect persisted even after 7 consecutive days of drug treatment (500 μg, twice daily) (FIG. 3, lower panel). As with rabbits, the compound was well tolerated.

The compositions of the present invention comprise propentofylline and a pharmaceutically acceptable vehicle. As used herein, the term “pharmaceutically acceptable vehicle” refers to any formulation that is acceptable, i.e., is safe and provides the appropriate delivery of an effective amount of propentofylline for the desired route of administration. The compositions of the present invention may be administered orally, or they may be administered locally to the eye via topical dosing or by a continuous release device placed in the cul-de-sac of the eye.

Propentofylline can be incorporated into a formulation, such as a tablet or a capsule, for oral administration. Hence, 100-1000 mg of propentofylline may be combined with inactive ingredients such as starch, lactose and magnesium stearate and formulated according to procedures known to those skilled in the art of tablet or capsule formulation. An example of a tablet formulation is shown in Example 4. This formulation will be administered to patients 1 to 6 times daily, 1 to 3 tablets each time.

Propentofylline can be incorporated into various types of ophthalmic formulations for delivery to the eye. The compound may be combined with ophthalmologically acceptable preservatives, surfactants, viscosity enhancers, penetration enhancers, buffers, sodium chloride, and water to form an aqueous, sterile ophthalmic suspension or solution. Ophthalmic solution formulations may be prepared by dissolving the compound in a physiologically acceptable isotonic aqueous buffer. Further, the ophthalmic solution may include an ophthamologically acceptable surfactant to assist in dissolving the compound. Furthermore, the ophthalmic solution may contain a thickener such as hydroxymethylcellulose, hydroxyethylcellulose, hydroxypropylmethylcellulose, methylcellulose, polyvinylpyrrolidone, or the like, to improve the retention of the formulation in the conjunctival sac. In order to prepare sterile ophthalmic ointment formulations, the active ingredient is combined with a preservative in an appropriate vehicle, such as, mineral oil, liquid lanolin, or white petrolatum. Sterile ophthalmic gel formulations may be prepared by suspending the active ingredient in a hydrophilic base prepared from the combination of, for example, carbopol-940, or the like, according to the published formulations for analogous ophthalmic preparations; preservatives and tonicity agents can be incorporated.

Propentofylline can also be incorporated in a continuous release system that will be placed in the cul-de-sac of the eye. Examples of such devices are shown by Zattaroni (U.S. Pat. No. 4,186,184). The release rate of propentofylline in this device will be 10 μg/hour to 1 mg/hour.

Propentofylline is preferably formulated as topical ophthalmic suspensions or solutions, with a pH of about 4.5 to 8.0. It will normally be contained in these formulations in an amount 0.1% to 5% by weight, but preferably in an amount of 0.2% to 3% by weight. An example of a topical ophthalnic formulation is presented in Example 5. Thus, for topical presentation 1 to 3 drops of these formulations would be delivered to the surface of the eye 1 to 4 times per day according to the routine discretion of a skilled clinician.

Propentofylline can also be used in combination with other agents for treating glaucoma, such as, but not limited to, β-blockers (e.g., timolol, betaxolol, levobetaxolol, carteolol, levobunolol, propranolol), carbonic anhydrase inhibitors (e.g., brinzolamide and dorzolamide), α₁ antagonists (e.g. nipradolol), α₂ agonists (e.g., iopidine and brimonidine), miotics (e.g., pilocarpine and epinephrine), prostaglandin analogues (e.g., latanoprost, travaprost, unoprostone, bimatoprost, and compounds set forth in U.S. Pat. Nos. 5,889,052; 5,296,504; 5,422,368; 5,688,819; and 5,151,444, “hypotensive lipids” (e.g., compounds set forth in U.S. Pat. No. 5,352,708), serotonergics, and neuroprotectants (e.g., compounds from U.S. Pat. No. 4,690,931, particularly eliprodil and R-eliprodil, as set forth in a pending application U.S. Ser. No. 06/203,350, and appropriate compounds from WO94/13275, such as, memantine.

### EXAMPLE 1

### Effect of Various Compounds on MMP-3 Expression in Cultured Human Trabecular Meshwork (HTM-35D) Cells

Human TM cells were isolated, characterized and cultured as described (Steely et al, The effects of dexamethasone on fibronectin expression in cultured human trabecular meshwork cells, Investigative Ophthalmology and Visual Science, 1992;33:2242-2250) They were maintained at 5% CO₂ and 37° C. in a medium consisting of Dulbecco's modified Eagle medium with Glutamax I supplemented with 10% fetal bovine serum and 50 μg/mL gentamicin. Confluent cell cultures were treated with the indicated compounds at the indicated final concentration for 72 hours and the medium assayed for proMMP-3 by a commercially available ELISA assay kit. (See FIG. 1.) Abbreviations: PPF-OH is hydroxypropentofylline.

### EXAMPLE 2

### Effect of Propentofylline on Outflow in Human Ocular Perfusion Organ Culture

Human ocular perfusion organ culture was performed as described (Tschumper, et al. Glycosaminoglycans of human trabecular meshwork in perfusion organ culture. Current Eye Research, 1990;9:363-369; Clark et al, Dexamethasone-induced ocular hypertension in perfusion-cultured human eyes. Investigative Ophthalmology and Visual Science 1995;36:478-489). Briefly, human cadaver eyes, 16 to 20 hours post mortem, were dissected at the equator and the lens, vitreous and iris were removed. The anterior segment of the eye, including cornea and sclera ring containing TM and ciliary body, was placed into a custom-made plexiglass culture dish and sealed in place with a plexiglass O-ring. Culture media (Dulbecco's modified Eagle medium) was placed in a reservoir and perfused through a central cannula in the bottom of the dish. The reservoir was raised to generate approximately 11 mmHg of hydrostatic pressure relative to the center of the perfused eye. The weight of the reservoir was recorded daily. Outflow rate was defined as the change in the weight of the reservoir per unit time.

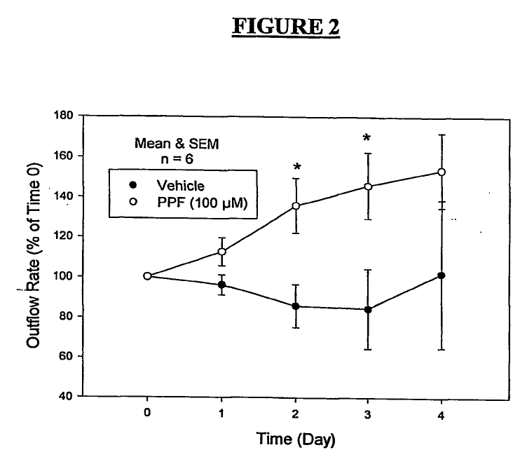

After a 2-4 days stabilization period, the eyes were perfused with either 100 μM propentofylline (PPF) or vehicle alone and their outflow rates monitored for is another 4 days. * represents p<0.05 by Student's t-test. (See FIG. 2.)

### EXAMPLE 3

### IOP-Lowering Effect of Propentofylline in Lasered Monkey Eyes

Top Panel: IOP-lowering effect of Propentofylline (PPF) (500 μg) in lasered monkey eyes. It clearly reduced IOP at 1 hour after topical ocular dosing.

Bottom Panel: Propentofylline was ocular hypotensive even after repeated doses. These monkeys received 500 μg of propentofylline twice daily for the indicated days and the IOP was measured right after the morning dosing. IOP of the eyes receiving vehicle control did not change (data not shown).

Studies were performed as described previously (Toris, et al., Aqueous humor dynamics in monkeys with laser-induced glaucoma, Journal of Ocular Pharmacology and Therapeutics, 2000;16:19-27). (See FIG. 3.)

### EXAMPLE 4

A tablet formulation suitable for oral administration, and useful for controlling intraocular pressure.

| Ingredient | Amount per Tablet (mg) |
|---|---|
| Propentofylline | 300 |
| Cornstarch | 50 |
| Lactose | 145 |
| Magnesium stearate | 5 |

### EXAMPLE 5

A topical ophthalmic composition useful for treating ocular hypertension:

| Ingredient | Concentration (% w/v) |
|---|---|
| Propentofylline | 1 |
| Dibasic Sodium Phosphate | 0.2 |
| Hydroxypropyl methylcellulose | 0.5 |
| Polysorbate 80 | 0.05 |
| Benzalkonium Chloride | 0.01 |
| Sodium Chloride | 0.7 |
| Edetate Disodium | 0.01 |
| NaOH/HCl | q.s., pH 7.4 |
| Purified Water | q.s. 100% |

1. A method for controlling intraocular pressure associated with glaucoma or ocular hypertension, which comprises administering a pharmaceutically effective dose of propentofylline.

2. The method of claim 1 which additionally comprises administering an additional agent which is for treating glaucoma or ocular hypertension.

3. The method of claim 1 wherein the propentofylline is administered orally.

4. The method of claim 3 wherein 100-1000 mg of propentofylline is orally administered.

5. The method of claim 1 wherein the propentofylline is administered topically to an eye.

6. The method of claim 5 wherein the propentofylline is delivered in a topical formulation at a concentration of 0.1-5 percent by weight.

7. The method of claim 1 wherein the propentofylline is delivered in a continuous release system placed in the cul-de-sac of an eye.

8. Use of propentofylline for the preparation of a pharmaceutical composition for controlling intraocular pressure associated with glaucoma or ocular hypertension.

9. The use according to claim 8 with an additional agent which is for treating glaucoma or ocular hypertension for the preparation of a pharmaceutical composition for treating glaucoma or ocular hypertension.

10. The use of claim 8 wherein the composition is an oral composition.

11. The use of claim 8 wherein the composition is a topical ophthalmic composition.
